(19) United States
(12) Patent Application Publication
(10) Pub. No.: US 2024/0238518 A1
(43) Pub. Date: Jul. 18, 2024

(54) SYSTEM FOR CONTROLLING AN ADMINISTRATION OF INFUSED SUBSTANCES

(71) Applicant: SOCIETE DE COMMERCIALISATION DES PRODUITS DE LA RECHERCHE APPLIQUÉE SOCPRA SCIENCES ET GÉNIE S.E.C., Sherbrooke (CA)

(72) Inventors: François LAMONTAGNE, St-Denis-de-Brompton (CA); Jean-Baptiste MICHAUD, Sherbrooke (CA); Alain GERVAIS, Saint-Lambert (CA); Félix CAMIRAND LEMYRE, Sherbrooke (CA)

(21) Appl. No.: 18/558,816

(22) PCT Filed: May 3, 2022
(86) PCT No.: PCT/CA2022/050684

§ 371 Date: Nov. 3, 2023

Related U.S. Application Data
(60) Provisional application No. 63/183,133, filed on May 3, 2021.

(51) Int. Cl. A61M 5/172; G16H 20/17; A61M 5/142

(52) U.S. Cl. CPC A61M 5/1723; G16H 20/17; A61M 2005/14208; A61M 2230/30

(57) ABSTRACT

A system for controlling an administration of a vasopressor agent comprising: at least one processing unit: and at least one non-transitory computer-readable memory communicatively coupled to the at least one processing unit and comprising computer-readable program instructions executable by the at least one processing unit for: receiving, by the at least one processing unit, a current arterial pressure of a patient: determining, with the at least one processing unit, based on a profile of the patient, a dose parameter to be adjusted as a function of at least the current arterial pressure and a target arterial pressure: and controlling, with the at least one processing unit, the operation of a pump administering the vasopressor agent as a function of the dose parameter: wherein the receiving, determining and controlling are performed continuously by the at least one processing unit.

### CROSS-REFERENCE TO RELATED APPLICATIONS

The present patent application claims the priority of U.S. Patent Application Ser. No. 63/183,133 filed on May 3, 2021, the content of which is incorporated herein by reference.

### TECHNICAL FIELD

The present disclosure pertains to equipment for the administration of medication, such as vasopressor medication or other infused medications or substances.

### BACKGROUND

Vasopressors (a.k.a. vasopressor medication, vasopressor drugs) are vasoactive agents used with patients having hemodynamic instability, for instance with a prevalent hypotension condition. Vasopressors may therefore be used to control the systemic vascular resistance to increase blood pressure. Examples of vasopressors include epinephrine, phenylephrine, norepinephrine, and dopamine, among others. Vasopressors may increase arterial pressure by inducing a constriction of blood vessels.

The administration of vasopressors must be balanced to overcome an hypotension condition without excessive vasoconstriction, as the latter may cause side effects such as ischemic injury to various organs, excessive demand on the heart muscle, etc. Accordingly, the infusion of vasopressors must be closely and regularly monitored by attending personnel so as to ensure as much as feasible that the arterial pressure remains within a target range.

It is known that arterial pressure will continuously vary. For practical reasons, it is not often feasible to have personnel attend to a patient and continuously adjust the flow of infusion in real time of vasopressor medication. In order to avoid low blood pressure, one common practice may be to increase a vasopressor dose and accept that the arterial pressure can exceed given targets, with possible deleterious effects to patients.

Hence, while risks associated with vasopressor administration and their adverse effects may vary across patients, as a function of age, comorbidities, patient frailty, current administration practices may nevertheless fail to personalize vasopressor therapy. Other current infusion therapy practices may have similar shortcomings.

### SUMMARY

In a first aspect, there is provided a system for controlling an administration of a vasopressor agent comprising: at least one processing unit; and at least one non-transitory computer-readable memory communicatively coupled to the at least one processing unit and comprising computer-readable program instructions executable by the at least one processing unit for: receiving, by the at least one processing unit, a current arterial pressure of a patient; determining, with the at least one processing unit, based on a profile of the patient, a dose parameter to be adjusted as a function of at least the current arterial pressure and a target arterial pressure; and controlling, with the at least one processing unit, the operation of a pump administering the vasopressor agent as a function of the dose parameter; wherein the receiving, determining and controlling are performed continuously by the at least one processing unit.

Further in accordance with the first aspect, for example, the profile of the patient includes a dosage range for the dosage parameter.

Further in accordance with the first aspect, for example, the system further comprises a system management device configured for selectively setting the dosage range, the system receiving the dosage range selectively set by an operator via the system management device.

Further in accordance with the first aspect, for example, the system further comprises a switch operatively connected between the controller and the pump configured for selectively overriding the controlled operation of the pump the system receiving overriding instructions from an operator via the switch.

Further in accordance with the first aspect, for example, the switch is configured for selectively setting the dose parameter upon the controlled operation of the pump being overridden the system receiving a dose parameter setting from an operator via the switch.

Further in accordance with the first aspect, for example, the system includes adjusting the dose parameter as a function of a current measurement associated with the patient other than the current arterial pressure.

Further in accordance with the first aspect, for example, the system includes adjusting the dose parameter as a function of a point-of-care device measurement.

Further in accordance with the first aspect, for example, the system is communicatively coupled to a system management device, the system triggering an alarm via the system management device upon the current arterial pressure exiting a target pressure range inclusive of the target arterial pressure.

Further in accordance with the first aspect, for example, the system is communicatively coupled to a database and the receiving of the arterial pressure is via the database.

Further in accordance with the first aspect, for example, the system further comprises an administration assistance system communicatively coupled to the at least one processing unit, wherein the computer-readable program instructions are updated by the administration assistance system based on the profile of the patient.

Further in accordance with the first aspect, for example, the system further comprises a patient monitoring device communicatively coupled to the at least one processing unit to send the current arterial pressure of the patient to the at least one processing unit.

Further in accordance with the first aspect, for example, the system further comprises the pump, the pump being operatively connected to the at least one processing unit.

Further in accordance with the first aspect, for example, the at least one processing unit and the at least one non-transitory computer-readable memory form a controller module of the system.

In a second aspect, there is provided a method of controlling an administration of a vasopressor agent comprising: receiving, with at least one processing unit, a current arterial pressure of a patient; determining, with the at least one processing unit based on a profile of the patient, a dose parameter to be adjusted as a function of at least the current arterial pressure and a target arterial pressure; and controlling, with the at least one processing unit, an administration of the vasopressor agent as a function of the dose parameter; wherein the receiving, determining and controlling are performed continuously by the at least one processing unit.

Further in accordance with the second aspect, for example, the determining of the dosage parameter includes limiting the dosage parameter to within a dosage range.

Further in accordance with the second aspect, for example, the method further comprises determining the dosage range.

Further in accordance with the second aspect, for example, the controlling of the administration of the vasopressor agent is overridable.

Further in accordance with the second aspect, for example, the method further comprises receiving a current measurement associated with the patient other than the current arterial pressure, the determining of the dose parameter to be adjusted as a function of the current measurement.

Further in accordance with the second aspect, for example, the dose parameter is adjusted by an initial impulse as a function of a difference between the target arterial pressure and the current arterial pressure.

Further in accordance with the second aspect, for example, an amplitude of the initial impulse is proportional to the difference between the target arterial pressure and the current arterial pressure.

Further in accordance with the second aspect, for example, an onset of a decrease of the initial impulse occurs before the current arterial pressure reaches the target arterial pressure.

Further in accordance with the second aspect, for example, the target arterial pressure is within a target zone of arterial pressures, the onset of the decrease of the initial impulse occurring before the current arterial pressure reaches a lower threshold of the target zone.

Further in accordance with the second aspect, for example, the dose parameter is adjusted by an initial increase as a function of a difference between the target arterial pressure and the current arterial pressure while the current arterial pressure is less than the target arterial pressure.

Further in accordance with the second aspect, for example, the determining of the dosage parameter is performed according to a trained learning algorithm being part of the profile.

Further in accordance with the second aspect, for example, the method further comprises producing, with the at least one processing unit, the trained learning algorithm based on one or more of a previous arterial pressure of the patient, the profile, and data from a pooled database.

### DESCRIPTION OF THE DRAWINGS

Reference is now made to the accompanying figures in which:

FIG. 1A is a block diagram of a system for controlling an administration of vasopressor agent in accordance with the present disclosure;

FIG. 1B is an exemplary graph showing contemplated actions in controlling the administration of the vasopressor agent with the system of FIG. 1A;

FIG. 1C is another exemplary graph showing contemplated actions in controlling the administration of the vasopressor agent with the system of FIG. 1A;

FIG. 2A is a block diagram of a network of a plurality of the system of FIG. 1A for controlling an administration of vasopressor agent in accordance with the present disclosure;

FIG. 2B is a block diagram of a network of the system of FIG. 1A and a plurality of modules for controlling an administration of vasopressor agent in accordance with the present disclosure;

FIG. 3 is a block diagram of an administration assistance system for use with the system of FIG. 1A;

FIG. 4A is a flow chart of a method for controlling an administration of vasopressor agent in accordance with the present disclosure;

FIG. 4B is a flow chart of steps of the method of FIG. 4A according to some embodiments;

FIG. 5A is an exemplary graph showing contemplated actions in controlling the administration of the vasopressor agent with the system of FIG. 1A as a function of a first initial arterial pressure;

FIG. 5B is an exemplary graph showing contemplated actions in controlling the administration of the vasopressor agent with the system of FIG. 1A as a function of a second initial arterial pressure; and

FIG. 5C is an exemplary graph showing contemplated actions in controlling the administration of the vasopressor agent with the system of FIG. 1A as a function of a third initial arterial pressure.

### DETAILED DESCRIPTION

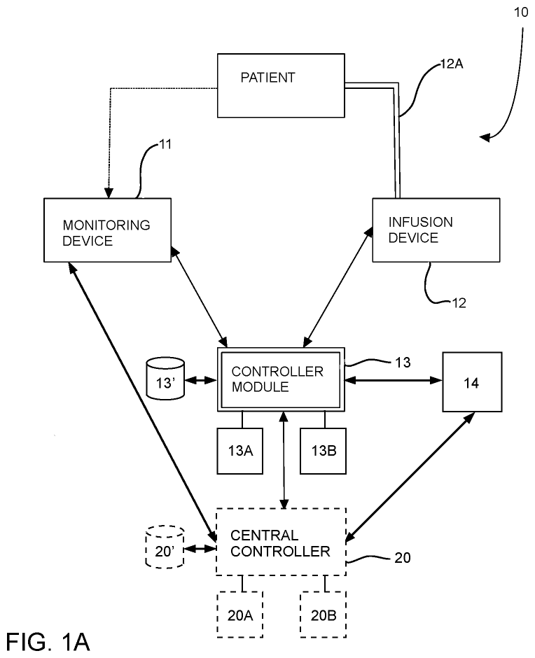

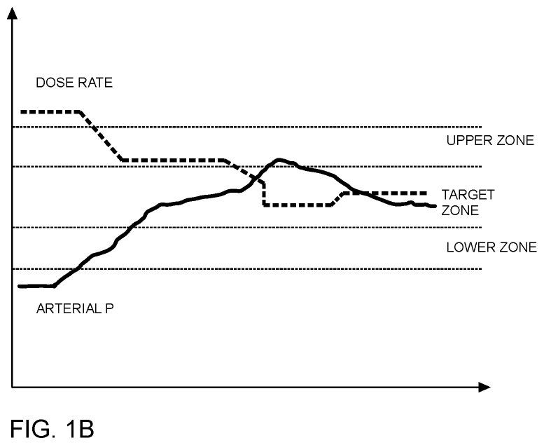

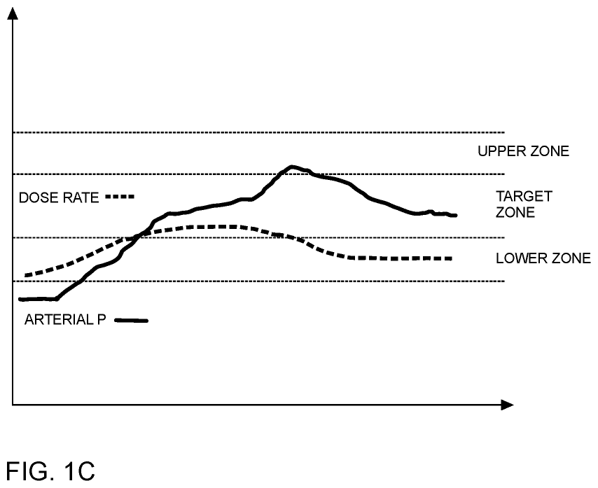

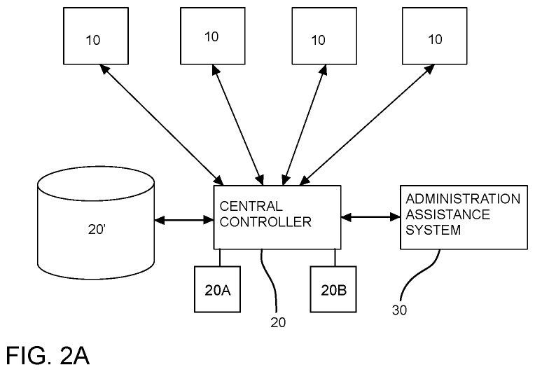

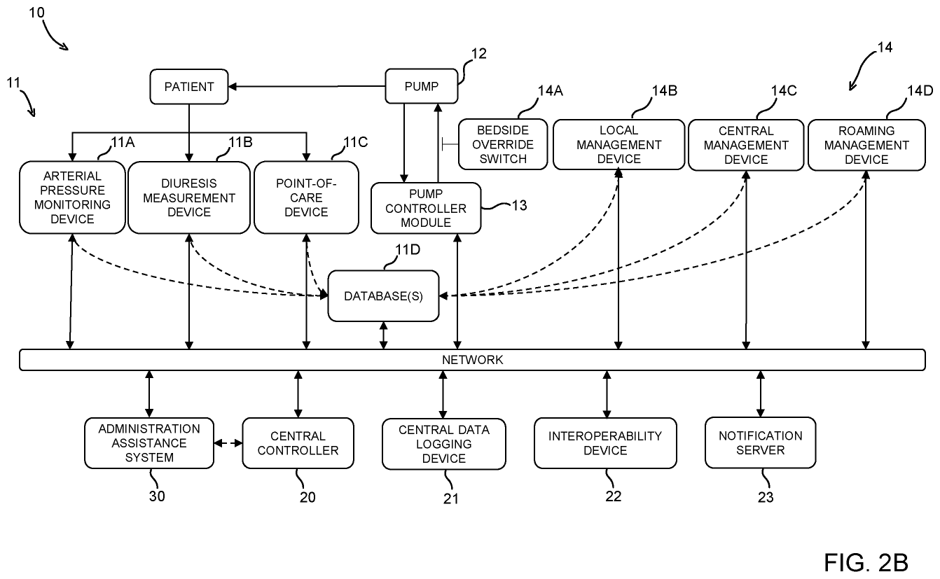

Referring to the drawings and more particularly to FIG. 1A, there is illustrated a system for controlling an administration of infused substances such as a vasopressor agent in accordance with the present disclosure at 10. For simplicity, the system will be referred to as the system 10 hereinafter. In an embodiment, there is one system 10 per patient. However, as detailed hereinbelow, it is considered a network of plurality of the systems 10 by way of a central controller 20. A central controller 20 may provide additional functions as described below with reference to FIGS. 2A and 2B. For example, a central controller 20 may be operated by a facility, such as a hospital, a network of hospitals, a network of institutions. The central controller 20 may be a single point access to all of the controller modules 13 of a network, as a possibility. The central controller 20 may collect patient data, and be a repository of patient medical files, keeping a log of the operation of the system 10. The system 10 has a patient monitoring device 11, an infusion pump 12 and a controller module 13 used in a closed loop with the patient monitoring device 11 and the infusion pump 12. As will become apparent from the forthcoming, depending on the embodiment, determinations and/or calculations according to which the pump 12 is controlled are performed by the controller module 13 and/or by the central controller 20 (if one is provided). A system management device 14 may also be provided so that parameters of the system 10 may be monitored and/or modified by an operator. The system 10 may thus, in some embodiments, be manually adjustable and/or overridable, notably to set alarm thresholds. The system management device 14 may also be known as a user interface, and may take various forms such as an integrated keyboard and screen, a portable device (e.g., smart phone, tablet), a remote control, etc.

In embodiments of the system 10 provided for administration of a vasopressor agent, the patient monitoring device 11 is of the type that can continuously monitor at least the arterial pressure of the patient, such as systolic and diastolic pressure. The patient monitoring device 11 may thus be said to include at least an arterial pressure monitoring device, or module. However, depending on the embodiment and on the infusion medication to be administrated, other vital signs of the patient may be monitored by the patient monitoring device 11, such as the heart rate, electrocardiogram (ECG), temperature, respiratory rate, blood oxygen saturation, etc. The patient monitoring device 11 has appropriate patient sensors to monitor the vital signs, such as a sphygmomanometer (i.e., blood pressure cuff), an intra-arterial canula or catheter, ECG sensors, spO2 sensors, diuresis measurement apparatus, etc. The patient monitoring device 11 may be a standalone device, such as a bedside device, with a display for providing vital sign data locally. For instance, the patient monitoring device 11 is a dedicated monitoring device, as an example among others. As another possibility, the monitoring device 11 is part of another apparatus, that may serve another function. For example, the monitoring device 11 may be part of a mechanical ventilator that is equipped with a CO₂ sensor, and other sensors (e.g. sphygmomanometer). The system 10 may use the arterial pressure signals from the other apparatuses, as the administration of a vasopressor agent may have an impact on the function of other organs (e.g., urinary output, oxygen levels, hear rate, etc). The system 10 may use signals indicative of vital signs other than arterial pressure, as such signals may be found by the system 10 to be clinically relevant to an ongoing vasopressor treatment episode. For example, such signals may be indicative of a projected variation of the arterial pressure, or of a contraindication to vasopressor treatment. As will become apparent from the forthcoming, the system 10 may be modular. In embodiments, the patient monitoring device 11 may correspond to at least one patient input module that provides input(s) to the system 10 based on which the system 10 may output suitable vasopressor dosage. Indeed, various patient input modules may be suitable for obtaining and/or communicating information (i.e., signal(s) and/or data) that is associated with the patient and from which the arterial pressure, whether contemporaneous or projected, may be derived. Depending on the embodiment, a plurality of patient input modules may be arranged in the closed loop at the onset of a vasopressor treatment episode. The number of patient input modules in the closed loop may also vary over the course of a vasopressor treatment episode. For example, a given patient input module may be intended to remain connected (e.g., an arterial pressure monitoring device, a diuresis measurement device, a wearable connected device worn by the patient), be intended for temporary connection (e.g., a sphygmomanometer) or be without direct connection (e.g., patient-specific databases, such as an electronic medical record, or pooled databases, such as biobanks) to the patient during a given treatment episode. A given patient input module may be a point-of-care (POC) measurement device, i.e., a device capable of measuring (or otherwise processing), at or near the location of the patient, a reading and/or a specimen obtained from the patient so as to provide patient data in a timely manner, i.e., for the patient data to be made available for consideration by the system 10 in the control of the administration of vasopressor agent. Patient data obtainable from suitable POC measurement devices which may be taken into account by the system 10 may pertain to one or more of blood gas, lactate, blood electrolyte(s), blood glucose, hemoglobin, rapid coagulation, creatinine, rapid cardiac marker(s), drug(s), pregnancy status, infectious disease(s) and various other bioassays, including biobank information related to genetic or epigenetic trait(s), for example single-nucleotide polymorphism (SNP) identification associated with idiosyncrasic vasopressor responsiveness, among other possibilities. Hence, the patient monitoring device 11 may be said to represent an arrangement of clinically relevant data sources available at a certain point in time during a treatment episode and from which the system 10 may derive the suitable vasopressor dosage for a given patient.

The infusion pump 12 may be a volumetric intravenous infusion pump, that is automatically operated in order to infuse a medication agent to a patient. The infusion pump 12 may also be said to be controllably operated, either automatically and/or with operator intervention when necessary or if desired. The infusion pump 12 may have the appropriate intravenous system 12A, or other administration mechanism, by which the medicinal agent is administered to the patient. In an embodiment, the intravenous system 12A for perfusion of the vasopressor into the patient includes a line (e.g., tubing), a needle or syringe, medication bag or other source of the vasopressor, valves and connectors, etc, as examples among others. The infusion pump 12 has the capacity to control the amount of medicinal agent injected, such as dose rate, dose over time, etc. The infusion pump 12 may be equipped with a display, which may be a module of the system management device 14, to locally adjust parameters of operation of the infusion pump 12, and override any remote control.

Both the patient monitoring device 11 and the infusion pump 12 may have telecommunication capacity, through wireless or wired connection, with data transmission technologies such as WiFi, Bluetooth®, as examples among others. Appropriate arrangements are taken for the control of the device 11 and pump 12 to be exclusive to the controller module 13, or dedicated staff manually operating the system 10 for example via the system management device 14. In an embodiment, the device 11 (or a module thereof), the pump 12 and the controller module 13 are a single device, or in a single casing, but it is contemplated to have distinct devices as well. For example, the controller module 13 may be embodied by computer-readable program instructions in a non-transitory computer-readable memory of the pump 12 communicatively coupled to the processing unit of the pump 12. In an embodiment, the controller module 13 may be located distally from the device 11 and the pump 12, such as in another room (e.g., central computer, server room, command center, etc). A bedside or control center safety switch may be provided to override any command otherwise governing the operation of the pump 12. In this way, the system 10 may revert to a conventional approach of manually determining the dose administered. The switch may be a module of the system management device 14 or may be part of the pump 12 or of the controller module 13. In some embodiments, the pump 12, the controller module 13 and the switch are parts of a same device of the system 10. In an embodiment, an optocoupling is between the infusion pump 12 and the processor of the controller module 13 so as to isolate the computer from an electrical overload at the pump such as that from the use of a defibrillator, for instance. Moreover, in the event of a malfunction of the controller module 13, an operator may go into override mode.

The controller module 13 may be a module in a central computer, a dedicated device, a dedicated processor as part of a computer that controllably communicates with the patient monitoring device 11 and the infusion pump 12. In embodiments of the system 10 in which the determinations and/or calculations according to which the pump 12 is controlled are performed by the central controller 20, the controller module 13 may in essence act as a loop-closing relay between the patient monitoring device 11 and the infusion pump 12, both of which may be off-the-shelf products and not specifically configured for closed loop administration of a vasopressor agent. In an embodiment, the controller module 13 is a non-transitory computer-readable memory communicatively coupled to the processing unit and comprising computer-readable program instructions executable to perform functions described herein. In an embodiment, the controller module 13 may include both a processing unit 13A and a non-transitory computer-readable memory 13B communicatively coupled to the processing unit 13A and including computer-readable program instructions executable by the processing unit 13A. The controller module 13 may be associated with an acquisition database 13′ so as to gather information on the patient, or retrieve patient profile data. In FIG. 1A, the controller module 13 is shown as having an adjacent acquisition database 13′ but any appropriate database may be used, including a cloud-based database and/or a database associated with the patient monitoring device 11, in which case the acquisition database 13′ may be omitted.

The controller module 13 has the capacity of communicating with the patient monitoring device 11 and the infusion pump 12. The controller module 13 receives data from the patient monitoring device 11, for instance in the form of digital values of the vital signs. Accordingly, upon receiving this data, the controller module 13 may control the operation of the infusion pump 12 to vary the amount of medicinal agent administered to the patient. In an embodiment, the controller module 13 may determine, calculate, compute a dose parameter as a function of a plurality of variables. The adjustment may be done in real time or quasi-real time, and continuously or at high frequency. The variables may include time-dependent patient-specific variables, such as any of the vital signs measured by the patient monitoring device 11, including one or more of arterial pressure (i.e., blood pressure), heart rate, ECG readings and laboratory results, blood oxygen saturation, temperature, respiratory rate, and current or past medical conditions. The variables may also include non-time-dependent data pertaining to a profile of the patient, including age, sex, weight, height, chronic comorbidities, outcome of vasopressor treatment episode (e.g., recovery time, acute complications, intensive care treatment duration, mortality), laboratory results, prescription-related variables (e.g., dosage range of prescription-only medication), etc. As a function of these variables, the controller module 13 may also take into consideration a target arterial pressure, or a target range of arterial pressure, in comparison to the measured (current) arterial pressure. In an embodiment, the target arterial pressure, or a target range of arterial pressure are as a function of an hypotension condition, and may also be as a function of an hypertension condition. The target arterial pressure, or range thereof, may be part of the above-mentioned prescription-related variables.

The controller module 13 may then control an operation of or drive the pump 12 as a function of the determination or calculation of dose parameter based in the variables described above. The control of the pump 12 may include increasing a dosage rate, reducing a dosage rate, maintaining a dosage rate, so to as induce a variation, maintaining or adjustment of an arterial pressure, vis à vis the target arterial pressure, or the target range of arterial pressure. The controller module 13 may consequently operate a closed loop for the administration of the vasopressor agent based on patent vital signs and patient profile. As the patient profile may include a prescription of specific ranges of vasopressor agent dosage and/or arterial pressure, the system 10 may in such cases be said to apply the prescription in an automated manner. In an embodiment, the controller module 13 has the capacity of communicating with both the monitoring device 11 and the pump 12, while the monitoring device 11 and the pump 12 may remain independent from one another, for instance without the capacity to communicate with one another.

As mentioned hereinabove, a system management device 14 may be provided, via which an operator may access data associated with the operation of the system 10, select and/or adjust values for operational parameters of the system 10 and/or select an operational mode of the system 10. The operational parameters may include prescription-related variables, the target arterial pressure (and in some cases thresholds for the target range), and thresholds for other monitored vital signs such as heart rate and/or diuresis monitoring for example. The operational parameters may also include default values and rules (which may be patient-profile dependent) for automatically adjusting certain operational parameters from an associated default value to a patient-specific value. An exemplary rule may provide certain default target arterial pressure value(s) for certain patient age(s) or age range(s). Another exemplary rule may provide for the default arterial pressure to be automatically adjusted depending on whether or not a patient has a certain genetic or epigenetic trait, for example a certain SNP. The system management device 14 may be integral to the patient monitoring device 11, the infusion pump 12 and/or the controller module 13. At least in some embodiments, the system management device 14 is modular, i.e., includes one more system management modules, e.g., component(s) distinct from the patient monitoring device 11, the pump 12 and the controller module 13, such as a switch, a computer, a smart phone, a tablet or the like. Such system management modules may either be provided as an integral part of the system 10 or be configured for communicating with the system 10, for instance with an application. In some embodiments, additional system management module(s) may even be connected to the system 10, for example during an ongoing treatment episode should the need arise. In an embodiment, the patient monitoring device 11 (or a module thereof), the pump 12, the controller module 13 and the system management device 14 (or a module thereof) are a single device or in a single casing, but it is contemplated to have distinct devices as well. Stated otherwise, the system 10 may in some embodiments be an integral device, i.e., a device that integrates all components required for controlling an administration of vasopressor agent. In some such embodiments, the system 10 may nevertheless be configured for optional supplementation with patient monitoring, controller and/or system management module(s).

Referring to FIG. 1B, there is illustrated an exemplary graph showing contemplated actions in controlling the administration of the vasopressor agent by the controller module 13, based on the readings obtained from the patient monitoring device 11, such as the arterial pressure. FIG. 1B illustrates a Target Zone for the arterial pressure, in which it is desired to maintain the arterial pressure. Moreover, FIG. 1B also illustrates a Lower Zone and an Upper Zone, both adjacent to the Target Zone, but respectively below and above the Target Zone. The Zones may be patient specific and/or selected by the controller module 13 as a function of the patient variables, or selected by attending personnel (e.g., physician). In an embodiment, the controller module 13 operates to maintain the arterial pressure in the Target Zone. In another embodiment, the controller module 13 aims to maintain the arterial pressure in any one of the Lower, Target and Upper Zones, but may modify the dose rate as a function of the location of the arterial pressure in any one of the three Zones. Additional Zones may be added as described below.

Pressure thresholds defining the Zones may be associated with alarms. For instance, the controller module 13 may trigger a notification or alarm upon receiving an input signal indicative of the arterial pressure entering the Lower Zone, the Target Zone or the Upper Zone. In some such cases, the resulting alarm may be merely a notification indicating that a given Zone has been entered. A Working Zone, which for example may correspond to a combination of the Zones mentioned hereinabove, may be defined, inside which the system 10 is deemed likely to be able to lead or maintain the arterial pressure inside the Target Zone autonomously, i.e., in an automated manner. The controller module 13 may trigger an alarm upon the arterial pressure exiting the Working Zone, signalling to an operator that manual intervention, or override, may be desirable in order to steer the arterial pressure back toward the Working Zone. In some embodiments, the Target Zone corresponds to the Working Zone, and the Lower and Upper Zones are omitted. Suitable system management device 14 (or module(s) thereof) may for instance convey the alarms to the operator and/or be used by the operator to effect the manual interventions. In a variant, alarms may be triggered based on trends on the blood pressure and/or doses, even if they remain in the Working Zone.

In FIG. 1B, the arterial pressure plot line starts as being below the Lower Zone, in an hypotension condition for the patient. The dose rate plot line, for the vasopressor agent, is contemporaneous over the X-axis time scale, and shows relatively high dose rate, as controlled by the controller module 13, in light of the hypotension condition, in an attempt to quickly increase the arterial pressure. As a result, the arterial pressure increases. The controller module 13 may monitor the arterial pressure increase, and note that the arterial pressure enters the Lower Zone. The controller module 13 may trigger a reduction of the dose rate as a result, or as a result of a monitored increase in arterial pressure. The dose rate is shown as decreasing linearly as controlled by the controller module 13, but other decrease patterns may be used, such as incremental (stepped), logarithm, squared, modulating, etc.

When the arterial pressure is in the Target Zone, the controller module 13 may stabilize the dose rate, with a plateau being shown as an example of a constant dose rate. However, the controller module 13 may also vary the dose rate when the arterial pressure is in the Target Zone, with stepped, linear patterns of adjustment being examples among others. In an embodiment, the amplitude in variation of the dose rate is lesser when the arterial pressure is in the Target Zone. In another embodiment, the amplitude in variation of the dose rate is dependent on the rate of variation of the arterial pressure.

Still in FIG. 1B, as the arterial pressure reaches the Upper Zone, the controller module 13 lowers the dose rate, in a stepped manner, as a possibility among others, in an effort to lower the arterial pressure into the Target Zone. In essence, the actions of the controller module 13 aim at maintaining the arterial pressure (e.g., systolic pressure or the mean arterial pressure) in the Target Zone, as much as possible, if not continuously. The fine-tuned adjustments permissible by the continuous monitoring of the controller module 13 may allow a lower amplitude of pressure variation and/or enhanced stability in the arterial pressure. The controller module 13 may also take into consideration the time lag between dose administration and the effects on the body, in an effort to stabilize the arterial pressure in the Target Zone. The dose rates selected by the controller module 13 may be based on variables described above, as the variables may be indicative of a patient's anticipated response to the administration of vasopressor agent. Moreover, some particular conditions may also come into play, in addition to the Zones shown. For example, the heart rate may be monitored by the controller module 13. If the heart rate is above a desired level, while the arterial pressure is well suited, it may be considered to lower the dose rate to lower the heart rate.

Referring to FIG. 1C, there is illustrated another exemplary graph showing contemplated actions in controlling the administration of the vasopressor agent by the controller module 13, based on the readings obtained from the monitoring device 11, such as the arterial pressure. FIG. 1C illustrates a Target Zone for the arterial pressure, in which it is desired to maintain the arterial pressure, a Lower Zone and an Upper Zone respectively below and above the Target Zone.

In FIG. 1C, the arterial pressure plot line starts as being below the Lower Zone, in an hypotension condition for the patient. The dose rate plot line, for the vasopressor agent, is contemporaneous over the X-axis time scale, and shows an insufficient dose rate, and prompts an increase initiated by the controller module 13. As a result, the arterial pressure increases. The controller module 13 may monitor the arterial pressure increase, and note that the arterial pressure enters the Lower Zone. The controller module 13 may trigger a reduction of the dose rate as a result, or as a result of a monitored increase in arterial pressure. FIG. 1C shows an approach in which the intial dose rate is low, in an attempt to cause a gradual increase of the arterial pressure in response to the infusion of the dose.

When the arterial pressure is in the Target Zone, the controller module 13 may stabilize the dose rate, with a plateau being shown as an example of a constant dose rate. However, the controller module 13 may also vary the dose rate when the arterial pressure is in the Target Zone, with stepped, linear patterns of adjustment being examples among others. In an embodiment, the amplitude in variation of the dose rate is lesser when the arterial pressure is in the Target Zone. In another embodiment, the amplitude in variation of the dose rate is dependent on the rate of variation of the arterial pressure.

Still in FIG. 1C, as the arterial pressure reaches the Upper Zone, the controller module 13 lowers the dose rate, in a sloped manner, as a possibility among others, in an effort to lower the arterial pressure into the Target Zone. As the arterial pressure approaches a target value (i.e., a median value of the Target Zone), the dose rate may approach another plateau.

Stated differently, the system 10 is for controlling an administration of a vasopressor agent and may include a processing unit (such as the processing unit 13A of the controller module 13), and a non-transitory computer-readable memory (such as the memory 13B of the controller module 13) communicatively coupled to the processing unit and comprising computer-readable program instructions executable by the processing unit for: receiving, by the processing unit, a current arterial pressure of a patient; determining, with the processing unit, based on a profile of the patient, a dose parameter to be adjusted as a function of at least the current arterial pressure and a target arterial pressure; and controlling an operation of a pump administering the vasopressor agent as a function of the dose parameter. The operation of the pump is controlled by a controller module according to the determined dose parameter. The receiving, determining and controlling are performed continuously, and in an automated manner, without the constant assistance of a human operator. A human operator may have an option to override the actions of the controller module 13, for example via a system management module. As will be described hereinbelow, in embodiments, either one or both of the processing unit and the non-transitory computer-readable memory of the system 10 can be either nearby or remote relative to the patient, and either dedicated or shared relative to the system 10. In embodiments, the determining the dose parameter is performed according to an algorithm that is an output of an administration assistance system 30 (FIG. 2A), details of which will be provided hereinbelow.

Referring to FIG. 2A, it is observed that a plurality of the system 10 may be networked via a central controller 20. Whereas the controller modules 13 of each system 10 of a given network may be located nearby a corresponding patient, the central controller 20 may be remote, for example in a data room of the hospital in which the systems 10 are located or, as the case may be, in a data center shared by several hospitals respectively housing at least one of the systems 10 of a same network. The central controller 20 may have a processing unit 20A and a non-transitory computer-readable memory 20B communicatively coupled to the processing unit 20A and comprising computer-readable program instructions executable by the processing unit 20A. For example, the central controller 20 may be a server, a group of servers, a cloud-based service, with the controller modules 13 being operated jointly, as a possibility among others. Therefore, the central controller 20 may acquire data for all of the patients that are part of the network. In an embodiment, the central controller 20 may gather the data from the patients as in associated database 20′, so as to perform or assist in the determination and/or calculation of the dose parameters by the controller modules 13 based on patient data. In embodiments, the calculations and/or determinations of the dose parameters are performed at least in part by the central controller 20. In some such embodiments, the dose parameters, or pump control instructions associated thereto, are sent from the central controller 20 to the controller modules 13 for the controller modules 13 to control their respective pumps 12 accordingly. In an embodiment, the data acquired in the arrangement of FIG. 2A may be used to train the administration assistance system 30, that may contribute to the accuracy and precision of the calculations and/or determinations made by the controller modules 13 and/or the central controller 20. The central controller 20 may also collect data from concurrent apparatuses used on a patient, in addition to the system 10. For example, if a mechanical ventilator apparatus or like medical device is used on a patient, or a fluid bolus is administered to the patient, the central controller 20 may reconcile the concurrent effects of the system 10 and that of the other treatments, to isolate the effect of the various treatments, or to learn about the concurrent effects.

In FIG. 2B, another exemplary networked implementation of at least one system 10 with a central controller 20 is depicted. Although a sole system 10 and a sole central controller 20 are shown, the central controller 20 is typically used with multiple systems 10. Moreover, a plurality of central controllers 20 (for example one for each of the several hospitals respectively housing at least one of the systems 10 of the same network) may be networked together, with each central controller 20 being networked with one or more underlying systems 10.

In this example, the patient monitoring device 11 of the depicted system 10 includes a plurality of patient input modules. The range of possible types of patient input modules is vast. Exemplary types include, as previously discussed, an arterial pressure monitoring device 11A, a diuresis measurement device 11B, a point-of-care (POC) measurement device 11C, and one or more connected database(s) 11D. The connected database(s) 11D may contain relevant data such as the electronic medical record of the patient, or patient prescription. The connected database(s) 11D may also include one or more biobank(s) containing pooled and typically anonymised patient data. Among the data provided by the patient monitoring device 11, some data may be referred to as “static” (such as genetic and/or epigenetic data, among other possibilities, that may be stored in the electronic medical record) whereas some data is time dependent (i.e., provided in real time or near real time, such as the arterial pressure obtained via the arterial pressure monitoring device 11A). Patient input modules 11A, 11B, 11C, 11D may in some cases share data directly with the central controller 20. Alternatively, two or more of the patient input modules 11A, 11B, 11C, 11D may be interconnected so as to share data before such data is available for determination of the dose parameters. For example, any one of the arterial pressure monitoring device 11A, the diuresis measurement device 11B and the POC measurement device 11C may send data to one or more of the connected databases 11D, for example to the electronic medical record of a corresponding patient, via which the central controller 20 may access such data. It should be noted that time dependent data acquired via the patient monitoring device 11, which may or may not be used in real time or near real time by the central controller 20, may leave an associated record entry in the electronic medical record (or in some cases another connected database 11D). Such record entries of time dependent data may accrue into historical data associated with a given patient and/or treatment episode and may be considered static data. Based on the available data, the central controller 20 determines the dose parameters, and communicates with the controller module 13 to control its associated pump 12 accordingly. Concurrently, the central controller 20 may communicate with the patient monitoring devices 11 and controller modules 13 of other networked systems 10 associated with other patients.

In this example, the depicted system 10 is networked with a plurality of system management modules 14, including in this case a bedside override switch 14A, a local management device 14B, a central management device 14C and a roaming management device 14D. The bedside override switch 14A is a single-patient, or patient-dedicated, device (i.e., a device that operates at the level of a given system 10 for a given patient) allowing to disengage the automated control otherwise provided by the system 10, such that the pump 12 operates according to manually specified dosage parameters. The switch 14A may include an interface suitable for such input, or may selectively render operational an input interface of the pump 12. Among the system management modules 14, one or more may allow an operator to monitor and/or modify parameters of the system 10 that govern the automated control. For instance, such functionality may be provided in single-patient (e.g., the local management device 14B) and multi-patient (e.g., the central management device 14C, the roaming management device 14D) implementations. Conveniently, the local management device 14B, for example an interface integrated to the patient monitoring device(s) 11 and/or pump 12 a laptop computer, a tablet or the like, may be disposed bedside for use by an operator attending to the patient. The central management device 14C, for example a desktop computer, may be stationed at a control center of an intensive care ward where multiple patients may be monitored simultaneously. The roaming management device 14D, for example a tablet, a phone, a pager, a smart watch or the like, can be used by an operator anywhere as long as a suitable power souce and a connection to the network are provided. Data sent or received by the system management modules 14 may transit via the electronic medical record (or in some cases another connected database 11D) of the concerned patient, so as to leave an associated record entry.

The central controller 20 may be said to be part of a backend of the network, i.e., a remote infrastructure that supports the provision of the above-mentioned functionalities of patient-dedicated (e.g., the patient monitoring device 11, the pump 12, the controller module 13) and operator-facing (e.g., the system management device 14) networked components. The backend may include a central data logging device 21, which may be a database implemented for storing operational data that does not pertain to a particular patient. The backend may also include suitable interoperability device(s) 22 configured to translate or otherwise format signal(s) and/or data generated by a networked component to render the same recordable, exchangeable and/or usable as needed in the network. The backend may also include a notification server 23 as a means for relaying data, such as alerts, to relevant system management devices 14. For example, in some embodiments, the notification server 23 is configured for pushing notifications to the roaming management devices 14D via a suitable communication protocol, whether standardized (e.g., SMS) or proprietary, or to any other staff communication system by which medical staff can be readily informed. In embodiments, the central data logging device 21, the interoperability device 22 and/or the notification server 23 may be implemented on the central controller 20. Depending on the embodiment, the administration assistance system 30, which will now be described in greater detail, may be part of the backend.

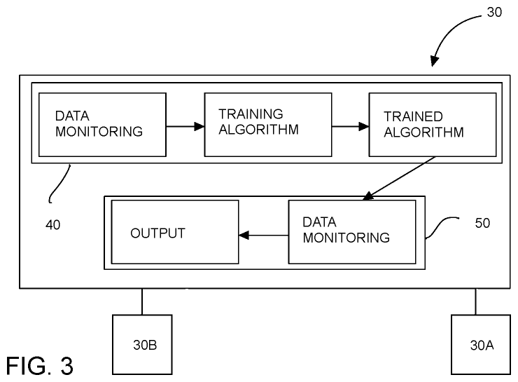

Referring now to FIG. 3, the administration assistance system 30 is shown in an exemplary embodiment, for instance as being processor unit 30A with a non-transitory computer-readable memory 30B communicatively coupled to the processing unit and comprising computer-readable program instructions executable by the processing unit 30A. In FIG. 3, the administration assistance system 30 is shown as having a machine-learning (ML) module 40 and an administration assistance module 50. In an embodiment, the machine-learning module 40 acquires the data from one or more of the central controllers 20, or directly from the controller modules 13, for training of the machine-learning module 40. The administration assistance module 50 may then contribute to the calculation or determination made by the controller modules 13, in adjusting the dose parameter(s) for the operation of the pump(s) 12, with patient specificity.

In a variant, the machine-learning module 40 receives monitoring data from the patient monitoring devices 11, controller module 13 and/or pump 12, for example along with the patient profile data, and all variables mentioned above and considered relevant. The data may be in the form of digital files, continuous signals, values, etc. Patient profile data acquired by the machine-learning module 40 may include age, weight, height, sex, race and ethnicity, body mass index, genetic and/or epigenetic conditions, pathologies, medical history, concurrent medications, allergies, family conditions, patient-specific administration history, and eventually, outcomes of the vasopressor administration episodes as set out above. Using this data acquisition, the machine-learning module 40 trains a learning algorithm to understand a patient's reaction to a dose being administered. The training of the learning algorithm may be based on training data (which may be patient data of any of the types discussed hereinabove) acquired from prior vasopressor administration episodes in patients treated with the system 10 or otherwise, such as data from patients being treated in more conventional methods, i.e., without the system 10. The interactions of the system 10 may then be adjusted as a function of the training.

Although the administration assistance system 30 is depicted in FIGS. 2A, 2B as being distinct from the controller module 13 and the central controller 20, it should be noted that the administration assistance system 30 may be implemented, mutatis mutandis, on one or more of the controller module(s) 13 and the central controller 20. The administration assistance system 30 may for example share at least one of a processing unit and a non-transitory computer-readable memory with the controller module 13 and/or the central controller 20 (if present). In one exemplary implementation, the computer-readable program instructions of the administration assistance system 30 may be stored on the non-transitory computer-readable memory 13B (FIG. 1A) to be executed by the processing unit 13A (FIG. 1A) of a given controller module 13. Alternatively, the administration assistance system 30 may be hosted on any suitable infrastructure, for example cloud-based or hosted on remote servers due to the data gathering functionalities of the administration assistance system 30. Such infrastructure may be computing farms, and may include hardware that is specifically adapted for machine learning applications, such as graphical processing units (GPUS) and/or tensor processing units (TPUs).

The vasopressor administration episodes may include past or current episodes, recorded in sufficient number. By taking observations of a sufficient number of administration episodes, the administration assistance system 30 may identify standards or deviations from standards based on the afore-mentioned patient profile values of weight, height, gender, race and ethnicity, body mass index, genetic and/or epigenetic conditions, pathologies, medical history, concurrent medications, allergies, family conditions, patient-specific administration history, etc, as such patient profile may have an impact on a patient's reaction to a dose rate of a given vasopressor agent. Specific patient profile data may cause a deviation over standard procedures, as the system may anticipate that the patient's response will deviate from expected response (e.g., cardiogenic shock and/or bleeding).

As a consequence of the training of the learning algorithm, the machine-learning module 40 may produce and output a trained learning algorithm, or hemodynamic model. The learning algorithm may be based on various types of known machine learning algorithm, such as neural networks, among others. The trained learning algorithm may be said to form part of the patient profile.

Still referring to FIG. 3, the administration assistance module 50 may be updated with the trained learning algorithm in real time or near real time. During a vasopressor administration episode, the administration assistance module 50 may access data from the patient monitoring device 11, from the controller module 13 or from the central controller 20. Using the trained machine learning algorithm, and with the data specific to a patient, the administration assistance module 50 may propose dose parameter specific to the patient, with a view to efficiently adjust the vital signs of the patient, and/or take into consideration multiple variables to suggest a dose parameter with a view to improving the patient's condition. Stated differently, the administration assistance module 50 may contribute to setting more precise diagnosed infusion parameters, and may also serve as a diagnostic tool to indicate a worsening condition. Moreover, the administration assistance module 50 may filter out sensor values that may be sensor errors.

In embodiments, the machine-learning module 40 and the administration assistance module 50 may be implemented on separate devices referred to respectively as server and client devices. The trained learning algorithm may be produced by a server device before being deployed on a client device. A server device having the machine-learning module 40 may for example be a computer referred to as an engineering and model generation device, whereas a client device having the administration assistance module 50 may be a controller (for example the controller module 13 or the central controller 20, depending on the embodiment). Optionally, before deployment, the trained learning algorithm may be submitted to a validation process, which may be performed either automatically by the server device, manually by an operator, or semi-automatically (i.e., with some operator involvement during an otherwise automated process).

The system 10 of the present disclosure, for instance as optionally used as part of a network, and with the assistance of machine learning, allows the continuous, instantaneous and/or automatic adjustment of the infusion rate for vasopressors in order to maintain the arterial pressure within target ranges, such as by maintaining the lowest dose minimal to avoid hypotension and avoid the complications of excessive administration of vasopressors.

The system 10 of the present disclosure may use the administration assistance system 30 in the context of automating in real time the titration of vasopressors and hence reduce differentials between target values and current values for arterial pressure. In an embodiment, the system 10 includes a closed loop arrangement for administering the doses of vasopressors. The system 10 of the present disclosure can operate the titration of vasopressor doses within the closed loop.

The system 10 may result in the reduction of interventions required from staff and increase the time that the arterial pressure resides within the desired target range. Consequently, there may be a reduction of adverse effects on the human body, such as myocardial infarction. The system 10 may also be reduce the total exposure to the medication by automating the weaning.

The system 10 automatically monitors the vital signs and may adjust vasopressor pump output so as to document patient parameters vis-à-vis medication infusion. The granularity of the administration is increased with the system 10 of the present disclosure.

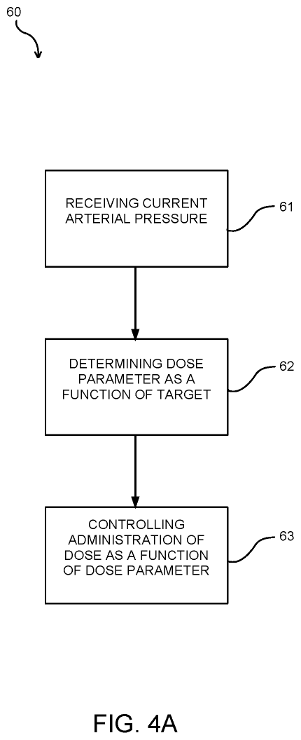

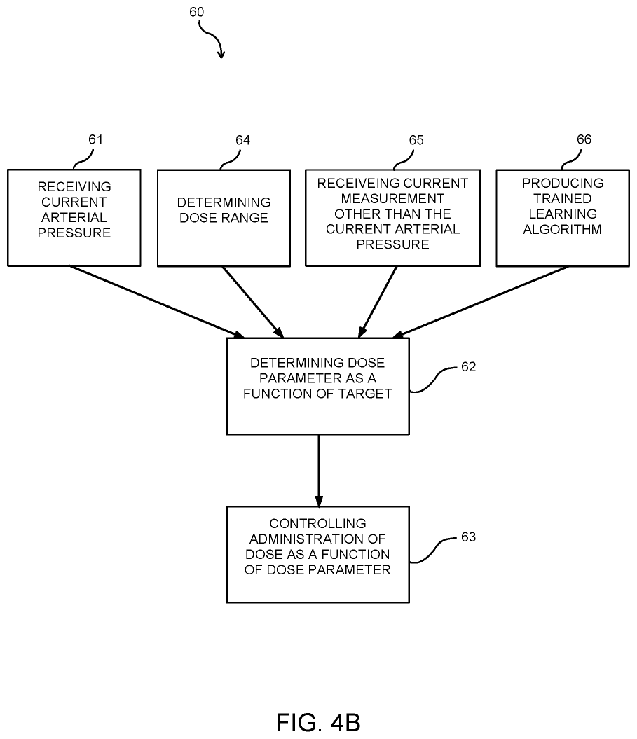

Referring to FIGS. 4A and 4B, a method 60 for example operated by the system 10 is for controlling an administration of a vasopressor agent in an automated manner. The method 60 may include steps such as; 61, receiving, by at least one processing unit, a current arterial pressure of a patient; 62, determining, with the at least one processing unit, based on a profile of the patient, a dose parameter to be adjusted as a function of at least the current arterial pressure and a target arterial pressure; 63, controlling, with the at least one processing unit, an operation of a pump administering the vasopressor agent as a function of the dose parameter. The receiving of 61, the determining of 62 and the controlling of 63 are performed continuously, and in an automated manner, without the constant assistance of a human operator.

In embodiments, the determining of the dosage parameter 62 includes limiting the dosage parameter to within a dosage range. In some such embodiments, the method 60 includes a step 64 of determining the dosage range, for example based on the profile. In embodiments, the controlling of the administration of the vasopressor 63 is overridable, for example via the switch 14A (FIG. 2B).

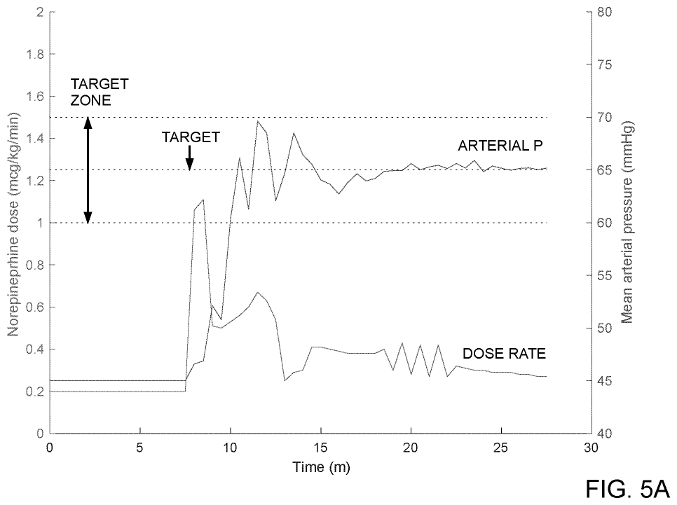

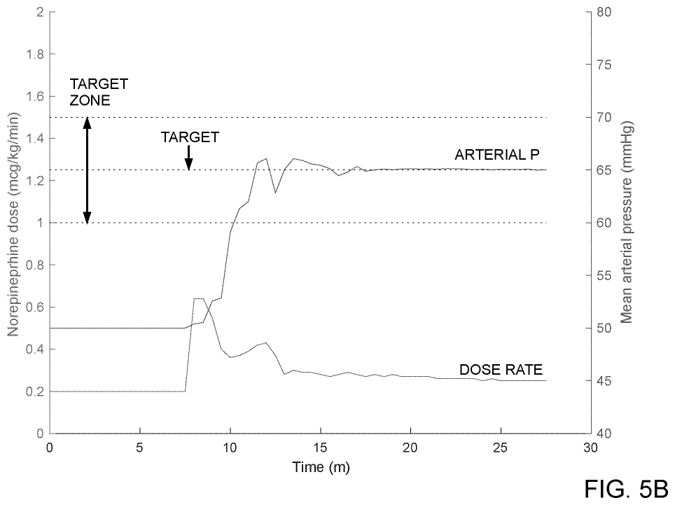

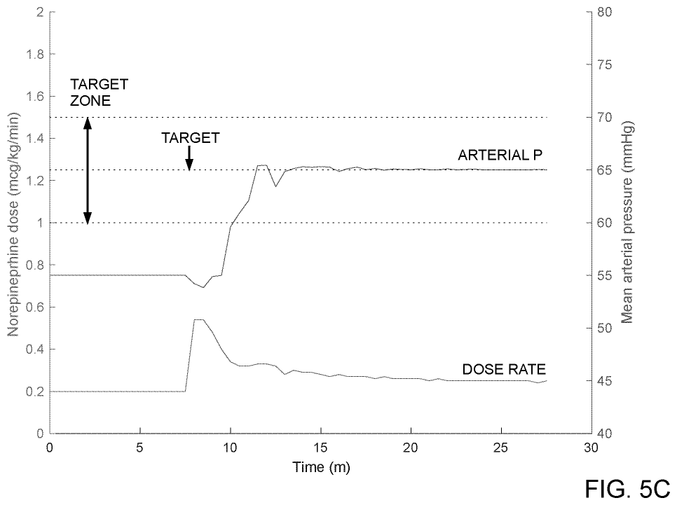

In embodiments, the method 60 includes a step 65 of receiving, with the at least one processing unit, a current measurement associated with the patient other than the current arterial pressure, in which case the determining of the dose parameter is to be adjusted as a function of the current measurement. The current measurement may be a diuresis measurement or any of the patient-specific variables mentioned hereinabove. In embodiments, the dose parameter may be adjusted by an initial impulse (such as seen in FIGS. 5A-5C and described hereinbelow) as a function of a difference between the target arterial pressure and the current arterial pressure. In some embodiments, an amplitude of the initial impulse is proportional to the difference between the target arterial pressure and the current arterial pressure. In some embodiments, an onset of a decrease of the initial impulse occurs before the current arterial pressure reaches the target arterial pressure. In some embodiments, the target arterial pressure is within a target zone of arterial pressures, the onset of the decrease of the initial impulse occurring before the current arterial pressure reaches a lower threshold of the target zone.

In embodiments, the dose parameter is adjusted by an initial increase as a function of a difference between the target arterial pressure and the current arterial pressure while the current arterial pressure is less than the target arterial pressure.

In embodiments, the determining of the dosage parameter 62 is performed according to a trained learning algorithm being part of the profile. In some embodiments, the method 60 includes a step 66 of producing, with the at least one processing unit, the trained learning algorithm based on one or more of a previous arterial pressure of the patient, the profile, and data from a pooled database.

Depending on the embodiment, the steps of the method 60 can be performed via one or more processing units, for example the processing unit 13A of the controller module 13, the processing unit 20A of the central controller 20, and the processing unit 30A of the administration assistance system 30.

While the system 10 described herein may contribute to the personalization of vasopressor administration episodes, and may also potentially reduce any adverse effect, some safety features may be present for a skilled human operator, such as a physician, nurse, etc, to override the actions of the controller module 13.

With reference to FIGS. 5A-5C, three exemplary vasopressor administration episodes will now be described as a function of three datasets respectively corresponding to a same hypothetical patient, the three datasets being initially identical except for their respective initial arterial pressures. It bears mentioning that the three exemplary vasopressor administration episodes are merely provided for illustrative purposes, and are patient dependent. Each initial arterial pressure may be a mean arterial pressure determined over a certain period of time, for example before any administration has occurred. Likewise, arterial pressure values shown on the graphs may be averaged over time periods and/or filtered to discard fault readings. In FIGS. 5A-5C, a dose rate (for example corresponding to the dose parameter determined by the processing unit of 13, 20 and/or 30 or to an effective dose rate measured at the pump 12) and an arterial pressure (for example measured by the patient monitoring device 11) expressed as a mean arterial pressure (MAP), are plotted as a function of time expressed in minutes. Under certain circumstances, a target arterial pressure (hereinafter, “Target”) suitable for the hypothetical patient may be for example of about 65 mmHg. Upper and lower pressure thresholds of a suitable Target Zone may be respectively of +5 mmHg relative to the Target. Other pressure target and threshold values are possible. In some cases, a difference between the upper threshold and the Target and a difference between the Target and the lower threshold are different. The Target and/or either pressure threshold may be selectively set (for example via the system management device 14) and/or determined (by 13, 20 and/or 30) during a given episode. Suitable dosage ranges may be prespecified by treating teams (e.g. up to 2 μg/kg/min). Ranges may be selectively set and/or determined during a given episode. For example, the determination of the dosage range boundaries may be bound by certain patient characteristics. At the onset of the episodes depicted in FIGS. 5A, 5B and 5C, the initial arterial pressures are of about 45 mmHg, of about 50 mmHg and of about 55 mmHg, respectively. Such disparity in initial arterial pressure may exist before enabling the closed-loop system, and may depend on the patient characteristics such as severity of illness, fluid status, comorbidities, etc. At the onset, the dose administered by the pump 12 may be insufficient, as illustrated in all example cases as 0.2 μg/kg/min. Under this initial dose, the arterial pressure is below the Target Zone. When the closed-loop system is enabled, in order to sway the arterial pressure toward the Target Zone, the system 10 may modulate the dosage by a suitable initial dose, which may include transient increases of the dosage, such an initial impulse. The initial dose may be determined in real time or near real time, or may be pre-determined, at least in part. Indeed, parameter(s) of the initial dose, such as amplitude of the initial impulse (i.e., a difference between a maximum value of the initial impulse and the initial arterial pressure) may be pre-determined. For example, the determination of the initial dose may be bound by certain values in presence of a certain patient characteristic, for example a genetic/epigenetic trait, in the patient profile. The initial dose may be determined as a function of a difference between the target arterial pressure and the initial arterial pressure (or the arterial pressure sensed during the onset period), i.e., an initial hypotension. In comparing FIGS. 5A-5C, it will be appreciated that the initial hypotension in FIG. 5A is greater than the initial hypotension in FIG. 5B, and the initial hypotension in FIG. 5B is greater than the initial hypotension in FIG. 5C. The amplitude of the initial impulse in FIG. 5A is greater than the amplitude of the initial impulse in FIG. 5B, and the amplitude of the initial impulse in FIG. 5B is greater than the amplitude of the initial impulse in FIG. 5C. In some embodiments, the amplitude of the initial impulse is proportional to the initial hypotension. It should also be noted that the initial impulse is defined by an increase in the dose followed by a decrease in the dose. The onset of the decrease may occur before the arterial pressure enters the Target Zone. This may in some cases prevent the arterial pressure from overshooting the Target Zone. Following the initial impulse, an onset of a stabilisation period is defined upon the arterial pressure entering the Target Zone. During the stabilisation period, the arterial pressure may vary within the Target Zone without any alarm being triggered in 13, 20 or 30 and relayed via 14.

The embodiments described in this document provide non-limiting examples of possible implementations of the present technology. Upon review of the present disclosure, a person of ordinary skill in the art will recognize that changes may be made to the embodiments described herein without departing from the scope of the present technology. For instance, the system 10 and/or the method 60 could be provided, mutatis mutandis, for controlling the administration of fluids, nutrients and/or medication other than or in addition (e.g., concurrently) to vasopressor agents. Further modifications could be implemented by a person of ordinary skill in the art in view of the present disclosure, which modifications would be within the scope of the present technology.

1. A system for controlling an administration of a vasopressor agent comprising:
at least one processing unit; and
at least one non-transitory computer-readable memory communicatively coupled to the at least one processing unit and comprising computer-readable program instructions executable by the at least one processing unit for;
receiving, by the at least one processing unit, a current arterial pressure of a patient;
determining, with the at least one processing unit, based on a profile of the patient the profile including a lower pressure threshold and an upper pressure threshold, a dose parameter to be adjusted as a function of at least the current arterial pressure and a target arterial pressure, and further as a function of the current arterial pressure relative to the lower pressure threshold and the upper pressure threshold; and
controlling, with the at least one processing unit, the operation of a pump administering the vasopressor agent as a function of the dose parameter;
wherein the receiving, determining and controlling are performed continuously by the at least one processing unit.

2. The system of claim 1, wherein the profile of the patient includes a dosage range for the dosage parameter.

3. The system of claim 2, further comprising a system management device configured for selectively setting the dosage range, the system receiving the dosage range selectively set by an operator via the system management device.

4. The system of claim 1, further comprising a switch operatively connected between the controller and the pump configured for selectively overriding the controlled operation of the pump the system receiving overriding instructions from an operator via the switch.

5. The system of claim 4, wherein the switch is configured for selectively setting the dose parameter upon the controlled operation of the pump being overridden the system receiving a dose parameter setting from an operator via the switch.

6. The system of claim 1, including adjusting the dose parameter as a function of a current measurement associated with the patient other than the current arterial pressure.

7. The system of claim 1, including adjusting the dose parameter as a function of a point-of-care device measurement.

8. The system of claim 1, wherein the system is communicatively coupled to a system management device, the system triggering an alarm via the system management device upon the current arterial pressure exiting a target pressure range inclusive of the target arterial pressure.

9. The system of claim 1, wherein the system is communicatively coupled to a database and the receiving of the arterial pressure is via the database.

10. The system of claim 1, further comprising an administration assistance system communicatively coupled to the at least one processing unit, wherein the computer-readable program instructions are updated by the administration assistance system based on the profile of the patient.

11. The system of claim 1, further comprising a patient monitoring device communicatively coupled to the at least one processing unit to send the current arterial pressure of the patient to the at least one processing unit.

12. The system of claim 1, further comprising the pump, the pump being operatively connected to the at least one processing unit.

13. The system of claim 1, wherein the at least one processing unit and the at least one non-transitory computer-readable memory form a controller module of the system.

14.-25. (canceled)

26. The system according to claim 1, wherein determining the dose parameter includes modifying a dose rate as a function of a location of the current arterial pressure relative to the lower pressure threshold and the upper pressure threshold.

27. The system according to claim 26, wherein the lower pressure threshold and the upper pressure threshold delimit a lower zone and an upper zone respectively, with a target zone between the lower zone and the upper zone, and wherein modifying a dose rate includes modifying the dose rates as a function of the zones.

28. The system according to claim 1, wherein the lower pressure threshold and the upper pressure threshold are associated with a hypotension condition.

29. A network for controlling an administration of a vasopressor agent at a plurality of locations, comprising:
a plurality of the system according to claim 1;
a central controller comprising at least one processing unit, and at least one non-transitory computer-readable memory communicatively coupled to the at least one processing unit and comprising computer-readable program instructions executable by the at least one processing unit for;
receiving data from the plurality of the systems, and
outputting a suggested dose parameter specific to a patient associated with any one of the plurality of the system, for a specific one of the plurality of the system to control the operation of the pump based on the suggested dose parameter.

30. The network according to claim 29, wherein the central controller is further for training a machine-learning module from said data, whereby the suggested dose parameter is from a trained algorithm of the machine-learning module.
